(19) United States
(12) Patent Application Publication
(10) Pub. No.: US 2009/0304755 A1
(43) Pub. Date: Dec. 10, 2009

(54) PHARMACEUTICAL FORMULATION OF LOSARTAN

(76) Inventors: Raghu Rami Reddy Kasu, Maharashtra (IN); Dhanashree Mistry, Maharashtra (IN); Sunderraj Manvi, Maharashtra (IN); Vijaya Kumar Thommandru, Maharashtra (IN); Himadri Sen, Maharashtra (IN)

Correspondence Address: MERCHANT & GOULD PC, P.O. BOX 2903, MINNEAPOLIS, MN, 55402-0903, US

(21) Appl. No.: 12/091,852

(22) PCT Filed: Jan. 6, 2006
(86) PCT No.: PCT/IN06/00041

§ 371 Date: Sep. 12, 2008

(30) Foreign Application Priority Data
Oct. 27, 2005 (IN) 979/KOL/2005

(51) Int. Cl. A61K 9/00; A61K 31/5415; A61P 9/12

(52) U.S. Cl. 424/400; 514/224.2

(57) ABSTRACT

A pharmaceutical composition comprising losartan and pharmaceutically acceptable salts thereof and a process of forming the same. The pharmaceutical composition of losartan comprises an active agent comprising an effective amount of losartan or its pharmaceutical salt thereof, and pharmaceutically acceptable additives, wherein d(0.9) of losartan is less than 50μ and/or specific surface area is more than 0.6 m²/gm. The process of preparation of pharmaceutical composition of losartan, comprises the steps of blending the losartan having d(0.9) less than 50μ and/or specific surface area more than 0.6 m²/gm, with the other intragranular excipients, dry compression, milling and screening to obtain granules, said granules being subsequently blended with extragranular excipients and compressed to tablets which is further coated.

### FIELD OF INVENTION

The invention relates to a pharmaceutical composition comprising Losartan and pharmaceutically acceptable salts thereof and a process of forming the same.

### BACKGROUND OF INVENTION

Losartan potassium is chemically described as 2-butyl-4-chloro-1-[p-(o-1H-tetrazol-5-ylphenyl)benzyl]imidazole-5-metahnol monopotassium salt. Its empirical formula is C₂₂H₂₂CIKN₆O, and its molecular weight is 461.01.

Losartan was first disclosed in U.S. Pat. No. 5,138,069, which also discloses use of this compound for hypertension, congestive heart failure and also chronic renal failure.

Losartan is known as angiotensin II receptor (Type AT1) antagonist used in hypertension alone or in combination with other antihypertensives, including diuretics; to reduce stroke and left ventricular hypertrophy in hypertensive patients excluding black patients, for nephropathy in type 2 diabetic patients.

Losartan potassium is currently available as tablets in different strengths of 100 mg, 50 mg and 25 mg.

The pharmacokinetics of losartan and its active metabolite, as mentioned in the pack insert of “Cozaar”, are linear with oral losartan doses up to 200 mg. Following oral administration, losartan is well absorbed; the systemic bioavailability of losartan is approximately 33%.

Losartan is also available as a combination product of losartan (50 to 100 mg) with hydrochlorothiazide (12.5-25 mg) is indicated for the treatment of hypertension and to reduce the risk of stroke in patients with hypertension and left ventricular hypertrophy.

The absorption of an active substance in the body following oral administration of a pharmaceutical formulation, particularly from the small and large intestine, is affected primarily by dose size/dissolution rate ratio, dissolution rate, degradation and metabolic conversion in the lumen and effective permeability or absorption of an active substance across the intestinal mucosa.

For an effective pharmaceutical formulation, it is of particular importance that the active substance in the region where drug absorption occurs undergoes rapid dissolution and obtains the highest possible concentration gradient over the shortest possible time. The above-described facts impose the problem to pharmaceutical technologists regarding how to prepare a pharmaceutical formulation, which will facilitate the most efficient absorption of highly soluble and low permeable losartan.

Commercially available losartan under the brand name of “COZAAR®” is a crystalline product. During our studies, we found that the bioavailability of amorphous losartan is dependent upon its particle size and specific surface area.

We have now surprisingly found that a careful manipulation of particle size and specific surface area of the active ingredient had an impact on the bioavailability of the drug.

### OBJECT OF THE INVENTION

According to the invention there is provided a pharmaceutical composition comprising

a) an active agent comprising an effective amount of amorphous losartan and its pharmaceutical salt thereof, and
b) pharmaceutically acceptable additives,
wherein d (0.9) of active agent is less than 50μ and/or specific surface area is more than 0.6 m²/gm.

Another object of the invention is to provide a process of preparation of a pharmaceutical composition of losartan, the said process comprising the steps of blending the losartan having d (0.9) less than 50μ and/or specific surface area more than 0.6 m²/gm, with the other intragranular excipients, dry compression, milling and screening to obtain granules, said granules being subsequently blended with extragranular excipients and compressed to tablets and coated.

Yet another object of the present invention is to provide a method of achieving bioequivalence between an immediate release coated tablets comprising amorphous losartan or pharmaceutically acceptable salt thereof and the commercially available immediate release tablets, the said tablet being marketed under the brand name of ‘COZAAR®’, the method comprising formulating the composition in the form of immediate release coated tablets wherein d (0.9) of active agent is less than 50μ and/or specific surface area is more than 0.6 m²/gm.

### DETAILED DESCRIPTION OF THE INVENTION

The invention relates to a pharmaceutical composition comprising

a) an active agent comprising an effective amount of amorphous losartan and its pharmaceutical salt thereof, and
b) pharmaceutically acceptable additives,
wherein d (0.9) of active agent is less than 50μ and/or specific surface area is more than 0.6 m²/gm.

“Pharmaceutical composition” means solid oral formulations, which includes but are not limited to, tablets, film coated tablets, granules, capsules, pellets, spheroids, microspheres, beads and the like.

“Losartan” is amorphous.

“Diuretic” include thiazide diuretic, wherein thiazide diuretic comprises HCTZ.

“Pharmaceutically acceptable salts” include potassium salt of losartan.

“d (0.9)” denotes a particle size wherein 90% (volume) particles have diameter less than the specified diameter d.

“Pharmaceutically acceptable additives” include the ingredients suitable for the preparation of a solid pharmaceutical formulation of present invention comprising diluents or fillers, binders, disintegrating agents, glidants, surfactants, lubricants and the like.

Fillers or diluents, include, but are not limited to confectioner's sugar, compressible sugar, dextrates, dextrin, dextrose, fructose, lactitol, mannitol, sucrose, starch, lactose, dibasic or tribasic calcium phosphate, calcium sulphate, calcium carbonate, xylitol, sorbitol, talc, micro-crystalline cellulose and the like can be used.

Binders, include, but are not limited to, any celluloses eg: -alkylcelluloses such as methyl cellulose, ethyl cellulose; hydroxyalkylcelluloses such as hydroxypropylcellulose, low substituted hydroxypropylcellulose, hydroxypropyl methylcellulose, sodium carboxymethylcellulose or mixtures thereof; microcrystalline cellulose; starches such as potato starch, wheat starch, corn starch, pregelatinised maize starch; natural gums such as acacia, alginic acid, guar gum; polyethylene oxide; liquid glucose, dextrin; polyvinylpyrrolidone such as products known under the registered trade marks Avicel, Filtrak, Heweten or Pharmacel can be used.

Glidants, include silicon dioxide, colloidal silica, powdered cellulose, talc, tribasic calcium phosphate and the like can be used.

Lubricants may be selected from those conventionally known in the art such as Mg, Al, Zn or Ca stearate, stearic acid, sodium stearyl fumarate, PEG, glyceryl behenate, hydrogenated vegetable oil, mineral oil light, and talc.

Disintegrants, include but are not limited to, cross linked polyvinyl pyrrolidone (crospovidone Polyplasdone Kollidon XL); starches and modified starches such as maize starch, pregelatinized starch, dried starch, sodium starch glycolate; gums such as alginic acid, sodium alginate, guar gum; croscarmellose sodium; any cellulose products such as microcrystalline cellulose and its salts, microfine cellulose, low substituted hydroxypropylcellulose; ion exchange resin such as polacrilin potassium; most preferably crosslinked polyvinyl pyrrolidone, crospovidone, crossinked carboxy methyl cellulose and Ac-Di-Sol or mixtures thereof.

One or more of these additives can be selected and used by the skilled artisan having regard to the particular desired properties of the solid oral dosage form. The amount of each type of additive employed, e.g. glidant, binder, disintegrant, filler or diluent and lubricant may vary within ranges conventional in the art.

Losartan can further be combined with diuretics such as thiazide diuretics, e.g. hydrochlorothiazide (HCTZ).

In a preferred embodiment, there is provided a process of making the solid oral dosage forms as hereinabove described comprising the steps of

i) sifting and blending the active agent or agents and pharmaceutically acceptable additives,
ii) subjecting the blend to compaction/slugging to form coprimates
iii) converting the coprimates to form granules and
iv) blending the granules with pharmaceutically acceptable additives
v) compressing the granules of step iv to form the tablets.

The process is carried out in the absence of water, i.e. it is a dry granulation (compaction/slugging) method.

Compaction of the blend into coprimate may be carried out using a slugging technique or preferably, roller compaction. Roller compaction apparatus is conventional and essentially utilizes two rollers, which roll towards each other. Hydraulic ram forces one of the rollers against the other to exert a compacting force against the dry blend fed into the roller compactor via a screw conveyor system.

The compression of the granulates into tablet cores can be carried out in a conventional tabletting machine, eccentric tabletting machine or a rotary compression machine.

The tablets thus obtained were further coated or film coated by using any of the conventional coating techniques in the prior art such as pan or perforated pans, for example spray coating using a fluidized bed granulator, a centrifugal fluidized bed coater or a spray drier or coating with a rotary granulator etc.

These coats comprised of one or more excipients selected from the group comprising film formers or coating agents, opacifiers, taste-masking agents, colouring agents, antitacking agents and the like.

Coating agents or film formers, include, but are not limited to, polysaccharides such as maltodextrin; alkyl celluloses such as methyl or ethyl cellulose; hydroxyalkylcelluloses (e.g. hydroxypropylcellulose or hydroxypropylmethylcelluloses); polyvinylpyrrolidone, polyvinyl alcohol, copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non-aqueous systems as appropriate.

Additives can be included along with the film formers to obtain satisfactory films. These additives can include plasticizers such as dibutyl phthalate, triethyl citrate, polyethylene glycol polyethylene derivatives such as polysorbate 80 and the like. Antitacking agents include talc, stearic acid, magnesium stearate, colloidal silicon dioxide and the like.

Surfactants include polysorbates, sodium lauryl sulphate and the like.

Opacifying agents include titanium dioxide, ferric oxide, sunset yellow and the like. All these excipients can be used at levels well known to the persons skilled in the art. There now follows a series of examples, which serve to illustrate the invention and is in no way a limitation to the same.

### EXAMPLES

| | | | |
|---|---|---|---|
| Example | Example | Example | Example |
| I | II | III | IV |

| | | |
|---|---|---|
| Sr. No. | Ingredients | mg/Tablet |

Intragranular (IG)

| | | | | | |
|---|---|---|---|---|---|
| 1 | Losartan Potassium | 100.0 | 100.0 | 100.0 | 100.0 |
| 2 | Microcrystalline cellulose | 40.0 | 55.0 | 55.0 | 55.0 |
| 3 | Lactose | 95.5 | 72.0 | 73.5 | 63.0 |
| 4 | Pregelatinised Starch | 15.0 | — | — | — |
| 5 | Collodial Silicon Dioxide | 14.5 | 16.5 | 16.5 | 16.5 |
| 6 | Magnesium Stearate | 2.5 | 2.5 | 2.5 | 2.5 |
| 7 | Sodium Starch Glycol late | — | 6.0 | — | — |
| 8 | Crosscarmellose Sodium | — | — | 4.5 | — |
| 9 | Crosslinked PVP | — | — | — | 6.0 |

Extragranular (EG)

| | | | | | |
|---|---|---|---|---|---|
| 10 | Microcrystalline cellulose | 10.0 | 30.0 | 30.0 | 30.0 |
| 11 | Pregelatinised Starch | 15.0 | — | — | — |
| 12 | Collodial Silicon Dioxide | 4.5 | 6.0 | 6.0 | 6.0 |
| | Aerosil | | | | |
| 13 | Magnesium Stearate | 3.0 | 3.0 | 3.0 | 3.0 |
| 14 | Sodium Starch Glycol late | — | 9.0 | — | — |
| 15 | Crosscarmellose Sodium | — | — | 9.0 | — |
| 16 | Crosslinked polyvinyl | — | — | — | 18.0 |
| | pyrrolidone | | | | |
| | Total | 300 | 300 | 300 | 300 |

Coating

| | | | | |
|---|---|---|---|---|
| Opadry White (Hydroxypropyl | 9 | 9 | 9 | — |
| methyl cellulose, hydroxypropyl | | | | |
| cellulose, titanium dioxide) | | | | |
| Moisture impermeable Coating: | — | — | — | 9 |
| Contains ingredients same as | | | | |
| innovator such as Hydroxy | | | | |
| propyl Methyl Cellulose, | | | | |
| Hydroxy propyl Cellulose, | | | | |
| Titanium Dioxide, D&C yellow | | | | |
| No. 10 Aluminium Lake, FD&C | | | | |
| blue No. 2 Aluminium Lake etc. | | | | |
| Total | 309 | 309 | 309 | 309 |

### Example V

| | | |
|---|---|---|
| Sr. No. | Ingredients | mg/Tablet |

Intragranular (IG)

| | | |
|---|---|---|
| 1 | Losartan potassium | 100 |
| 2 | Hydrochlorothiazide (HCTZ) | 25 |
| 3 | Microcrystalline cellulose | 75 |
| 4 | Pregelatinized starch | 30 |
| 5 | Lactose | 169 |
| 6 | Colloidal silicon dioxide | 14.5 |
| 7 | Mg stearate | 2.5 |

Extragranular (EG)

| | | |
|---|---|---|
| 8 | Microcrystalline cellulose | 20 |
| 9 | Pregelatinised starch | 55 |
| 10 | Colloidal silicon dioxide | 4.5 |
| 11 | Mg stearate | 4.5 |
| 12 | Total | 500 mg |

Coating

| | |
|---|---|
| Opadry yellow | 15.0 |
| Total | 515 mg |

Brief Manufacturing Procedure:
1. Weigh all the ingredients as per the formula.
2. Sift all the intragranular ingredients and mix well. Lubricate these ingredients with magnesium stearate (IG).
3. Perform compaction/slugging of step 2. Mill the compacted/slugged material into granules and sifted to get desired size.
4. Lubricate step 3 with magnesium stearate (EG) and add sifted extragranular excipients for sufficient time and compress on suitable punches.
5. Coat the compressed material of step 4 using suitable coating material.

We have surprisingly found that manipulation of particle size and specific surface area of losartan in pharmaceutical compositions had impact on the bioavailability of losartan.

The results of our investigations have shown that it is advantageous to use losartan, with a particle size d (0.9) less than 50μ measured by Malvern light scattering technique, and/or a specific particle specific surface area more than 0.6 m²/g measured by the Gas adsorption analyzer method (BET).

Two formulations were made as described in example 1; one with losartan having d₉₀ less than 50μ (A) and the other having d₉₀ more than 50μ (B). These formulations were subjected to a dissolution study in water (Table 1), and bioequivalence study against commercially available losartan formulation COZAAR® (Table 2).

### Dissolution Study

The in vitro specifications for generic products should be established based on a dissolution profile. In the case of a generic drug product, the dissolution specifications are generally the same as the reference listed drug.

A comparative dissolution study was carried out in water. The following compositions were tested: immediate release tablets comprising of 100 mg of losartan potassium, comprising losartan with varying particle size and specific surface area (A and B), prepared according to example 1 as test and Cozaar® having losartan potassium 100 mg, by Merck as reference.

To determine the similarity between the dissolution profiles of the test and reference product a simple model independent approach, that is f₂ (similarity factor), was carried out. As per US FDA, f₂ values should lie between 50-100 for rendering two dissolution profiles similar. The results obtained are summarized below in Table 1.

TABLE 1
The f₂ values of dissolution profiles of the losartan potassium
immediate release tablets against commercially available immediate
release tablets “Cozaar ®” in water using USP apparatus II.

| | % Drug Release; 50 rpm | | |
|---|---|---|---|
| | | Example 1 | Example 1 |
| Time in min | Cozaar ® | with Losartan A | with Losartan B |
| 5 | 19 | 16 | 19 |
| 10 | 40 | 42 | 45 |
| 15 | 60 | 64 | 67 |
| 20 | 75 | 80 | 84 |
| 30 | 88 | 99 | 96 |
| 45 | 95 | 99 | 97 |
| f₂ | | 63 | 62 |

A - Physical parameters of losartan
d (0.1) = 0.869μ
d (0.5) = 10.077μ
d (0.9) = 21.382μ
Specific Surface Area = 0.7-0.8 m²/g
B - Physical parameters of losartan
d (0.1) = 1.806μ
d (0.5) = 24.812μ
d (0.9) = 53.931μ
Specific Surface Area = 0.4-0.5 m²/g

The above results clearly show that the f₂ values were acceptable (as per the limits of 50-100 as established by the US FDA for claiming similarity between the dissolution profiles of the test and reference product) and were not dependent upon the particle size and specific surface area of losartan. However, surprisingly when we subjected the two formulations to bioequivalence studies as given below, we found that the particle size and specific surface area play an important role in determining bioequivalence of losartan.

### Bioequivalence Study

A bioequivalence study was carried out in 12 healthy human volunteers receiving single dose of losartan in fed state using immediate release tablets comprising of 100 mg of losartan potassium, comprising losartan with varying particle size and specific surface area (A and B), prepared according to example 1, as test and COZAAR® having losartan 100 mg, by Merck, as reference.

Study was monitored in terms of the pharmacokinetic parameters Cₘₐₓ and AUC. AUCs are plots of plasma concentrations of losartan along the ordinate (Y-axis) against time on the abscissa (X-axis). Generally, the values for AUC represent a number of values taken from all the subjects in a population and are, therefore, mean values averaged over the entire population. Cₘₐₓ, the observed maximum in a plot of plasma level concentration of losartan (Y-axis) versus time (X-axis) is likewise an average value.

The ratios of the log transformed mean values for Cₘₐₓ and AUC for the test and reference product (T/R ratio) is a measure of the bioequivalence between the test and reference product. Values between 80 and 125% for the 90% confidence intervals of these ratios indicate bioequivalence as recommended by the US FDA.

Bioequivalence data for the losartan tablets against the commercially available tablets “COZAAR®” is shown below in Table 2.

TABLE 2
Fed BE study data of Losartan immediate release tablets against
commercially available immediate release tablets COZAAR ®; n = 12

| | Log Transformed T/R | Log Transformed T/R |
|---|---|---|
| | (%) of Geometric Least | (%) of Geometric Least |
| PK | Square Mean of Example 1 | Square Mean of Example 1 |
| Parameters | with Losartan A | with Losartan B |
| Cₘₐₓ | 102.77 | 72.98 |
| AUC₍₀₋ₜ₎ | 101.51 | 100.81 |
| AUC_{(0-∞)} | 102.12 | 101.22 |

Cₘₐₓ = Maximum plasma concentration
AUC₍₀₋ₜ₎ = Area under the plasma concentration time curve from time 0 to t
AUC_{(0-∞)} = Area under the plasma concentration time curve from time 0 to ∞

We have surprisingly found that, although pharmaceutical formulation comprising losartan, having varying particle size and specific surface area, had similar in vitro dissolution profiles as that of the innovator product, but the pharmaceutical formulation (B) was not bioequivalent to the innovator product, where as the pharmaceutical formulation (A) was bioequivalent to the innovator product. As can be seen from the data above in Tables 2, a Log transformed T/R (%) ratio of Least Square mean in fed study, in case of Cₘₐₓ, AUC₍₀₋ₜ₎ and AUC_{(0-∞)} for pharmaceutical formulation A was well within the limits as established by the US FDA for claiming bioequivalence between a test and reference product.

Similar observations were found with pharmaceutical formulation comprising losartan (of varying particle size and specific surface area) and HCTZ combination.

1. A pharmaceutical composition of losartan comprising:
a. an active agent comprising an effective amount of losartan or its pharmaceutical salt thereof, and
b. pharmaceutically acceptable additives,
wherein d(0.9) of losartan is less than 50μ and/or specific surface area is more than 0.6 m²/gm.

2. A pharmaceutical composition of claim 1, wherein the losartan is amorphous.

3. Losartan and its pharmaceutical salt thereof, wherein d(0.9) of losartan is less than 50μ and/or specific surface area is more than 0.6 m²/gm.

4. Losartan of claim 3, wherein losartan is amorphous.

5. A pharmaceutical composition of claim 1, wherein the said composition is solid oral dosage form.

6. A pharmaceutical composition of claim 1, wherein losartan is present in a unit dose of 25 mg to 100 mg.

7. A pharmaceutical composition of claim 1, wherein the pharmaceutically acceptable additives are selected from the group comprising fillers or diluents, binders, lubricants, glidants and disintegrants.

8. A pharmaceutical composition of claim 1, wherein the diluent is one or more selected from the group comprising confectioner's sugar, compressible sugar, dextrates, dextrin, dextrose, fructose, lactitol, mannitol, sucrose, starch, lactose, dicalcium phosphate, calcium sulphate, xylitol, sorbitol, talc, microcrystalline cellulose or mixtures thereof.

9. A pharmaceutical composition of claim 1, wherein the binder is one or more selected from the group comprising methyl cellulose, ethyl cellulose, hydroxypropylcellulose, low substituted hydroxypropylcellulose, hydroxypropyl methylcellulose, sodium carboxymethylcellulose, potato starch, wheat starch, corn starch, microcrystalline cellulose, pregelatinised maize starch, liquid glucose, acacia, guar gum, alginic acid, dextrin, polyethylene oxide, polyvinylpyrrolidone or mixtures thereof.

10. A pharmaceutical composition of claim 1, wherein the lubricant is one or more selected from the group comprising stearic acid, Mg, Al, Zn or Ca stearate, polyethyleneglycol, glyceryl behenate, Mineral oil light, hydrogenated vegetable oil, sodium stearyl fumarate, talc or mixtures thereof.

11. A pharmaceutical composition of claim 1, wherein the glidant is one or more selected from the group comprising silicon dioxide, colloidal silica, powdered cellulose, talc, tribasic calcium phosphate or mixtures thereof.

12. A pharmaceutical composition of claim 1, wherein the disintegrant is one or more selected from the group comprising cross linked polyvinylpyrrolidone, maize starch, dried starch sodium starch glycolate, alginic acid, sodium alginate, guar gum, croscarmellose sodium, microcrystalline cellulose and its salts, microfine cellulose, low substituted hydroxypropylcellulose, ion exchange resin or mixtures thereof.

13. A process of preparation of a pharmaceutical composition of losartan, the said process comprising the steps of blending the losartan having d(0.9) less than 50μ and/or specific surface area more than 0.6 m²/gm, with the other intragranular excipients, dry compression, milling and screening to obtain granules, said granules being subsequently blended with extragranular excipients and compressed to tablets which is further coated.

14. A pharmaceutical composition of claim 1, is further coated wherein the coating layer comprises one or more excipients selected from the group comprising coating agents, plasticizers, antitacking agents, surfactants, coloring agents and opacifiers.

15. A pharmaceutical composition of claim 14, wherein the coating agent is one or more selected from the group comprising maltodextrin, methyl cellulose, ethyl cellulose, hydroxypropylcellulose, hydroxypropylmethylcelluloses, polyvinylpyrrolidone, polyvinyl alcohol, copolymers of vinylpyrrolidone, vinyl acetate, methacrylic acid polymers and alginate based coating.

16. A pharmaceutical composition of claim 14, wherein the plasticizer is selected from the group comprising dibutyl phthalate, triethyl citrate, polyethylene glycol and mixtures thereof.

17. A pharmaceutical composition of claim 14, wherein the antitacking agent is one or more selected from the group comprising talc, colloidal silicon dioxide, stearic acid, its salts and derivatives.

18. A pharmaceutical composition of claim 14, wherein the surfactant is one or more selected from the group comprising polysorbates, sodium lauryl sulphate and mixtures thereof.

19. A pharmaceutical formulation of losartan of claim 1, further comprises a thiazide diuretic such as hydrocholrothiazide.

20. A method of achieving bioequivalence between an immediate release losartan coated tablets prepared as per example 1, having 100 mg of losartan and a commercially available 100 mg immediate release tablets of losartan, the said tablet being marketed under the brand name of ‘COZAAR®’, the method comprising formulating the composition in the form of immediate release coated tablets wherein d(0.9) of losartan is less than 50μ and/or specific surface area is more than 0.6 m²/gm.
